(19) United States
(12) Patent Application Publication
(10) Pub. No.: US 2012/0136191 A1
(43) Pub. Date: May 31, 2012

(54) CATALYST AND PROCESS

(75) Inventors: Edmund Hugh Stitt, Billingham (GB); Michael John Watson, Eaglescliffe (GB); Lynn Gladden, Cambridge (GB); James McGregor, Hemingford Grey (GB)

(73) Assignees: CAMBRIDGE ENTERPRISE LTD., Cambridge, Cambridgeshire (GB); JOHNSON MATTHEY PLC, London (GB)

(21) Appl. No.: 13/376,301

(22) PCT Filed: Jun. 4, 2010
(86) PCT No.: PCT/GB2010/050944

§ 371 Date: Feb. 21, 2012

(30) Foreign Application Priority Data
Jun. 5, 2009 (GB) 0909694.2
Aug. 5, 2009 (GB) 0913579.9

(51) Int. Cl. C07C 5/333; B82Y 30/00

(52) U.S. Cl. 585/662; 585/661; 977/810; 977/902; 977/811

(57) ABSTRACT

The invention is a method of dehydrogenating a hydrocarbon, especially an alkane, to form an unsaturated compound, especially an alkene, by contacting the alkane with a catalyst comprising a form of carbon which is catalytically active for the dehydrogenation reaction. The catalyst may be formed by passing a hydrocarbon over a metal compound at a temperature greater than 650° C.

### CROSS-REFERENCE TO RELATED APPLICATIONS

This application is the U.S. National Phase application of PCT International Application No. PCT/GB2010/050944, filed Jun. 4, 2010, and claims priority of British Patent Application No. 0909694.2, filed Jun. 5, 2009, and British Patent Application No. 0913579.9, filed Aug. 5, 2009, the disclosures of all of which are incorporated herein by reference in their entireties for all purposes.

### FIELD OF THE INVENTION

The present invention concerns catalytic processes, especially but not exclusively for the dehydrogenation of hydrocarbon compounds, and catalysts used for such processes.

### BACKGROUND OF THE INVENTION

Catalytic dehydrogenation of hydrocarbon chains, especially alkanes, are important processes commercially for the production of unsaturated compounds. In particular the production of alkenes such as propene and butenes by dehydrogenation of the corresponding alkanes, i.e. propane and butane, form an important source of feedstocks for the manufacture of polyolefins and other products.

Processes for the dehydrogenation of alkanes are well known and widely used in industry. Non-oxidative dehydrogenation processes may be conducted using transition metal catalysts such as vanadia or chromia at temperatures of up to about 550° C. These catalysts deactivate rapidly under reaction conditions due to the formation of carbon deposits on the catalyst. The catalyst is periodically regenerated by burning off the carbon in an oxidation step. For example, GB-A-837 707 describes dehydrogenation of hydrocarbons employing a regenerable chromia catalyst wherein part of the chromia is oxidised to the hexavalent state during the oxidative regeneration process. The description indicates that the heat of combustion of the by-product carbon during the regeneration step can supply the heat required for the dehydrogenation reaction and that the reduction of the hexavalent chromium compound, which occurs during the reaction stage, can supplement the heat. This type of process is still widely used for the production of propene and butene but the requirement to regenerate the catalyst, typically after 20-30 minutes online, increases the cost and complexity of the process and the plant required. U.S. Pat. No. 5,087,792 describes an alternative process for the dehydrogenation of a hydrocarbon selected from the group consisting of propane and butane using a catalyst comprising platinum and a carrier material wherein the spent catalyst is reconditioned in a regeneration zone that uses, in the following order, a combustion zone, a drying zone and a metal re-dispersion zone to remove coke and recondition catalyst particles.

In U.S. Pat. No. 5,220,092 and EP-A-0556489, alkanes are dehydrogenated by contacting them with a catalyst containing vanadia on a support at elevated temperature for less than 4 seconds; a contact time of 0.02 to 2 seconds is said to give very good results. The alkanes are fed to the catalyst as short pulses interrupting a continuous flow of argon. A continuous regeneration of the catalyst for removal of coke, similar to the regeneration carried out in a fluidised catalytic cracking reaction, is preferred.

US-A-2008/0071124 describes the use of a supported nanocarbon catalyst for the oxidative dehydrogenation of alkylaromatics, alkenes and alkanes in the gas phase. This reference does not, however, describe or suggest that carbon nanostructures are stable and catalytically active for dehydrogenation reactions under non-oxidising conditions, i.e. in the absence of an oxygen-containing gas.

Processes for the oxidative dehydrogenation of alkanes are also practised using various metal oxide catalysts and mixed metal oxides. Such processes have the disadvantage that the oxidising conditions may cause the formation of oxygenated by-products such as alcohols, aldehydes, carbon oxides and also convert at least some of the produced hydrogen to water. There is a need for improved dehydrogenation processes, in particular for the production of lower alkenes such as propene and butene.

### SUMMARY OF THE INVENTION

According to the invention we provide a process for carrying out a chemical reaction comprising the step of passing a feed stream containing at least one reactant compound over a catalyst comprising a catalytically active carbon phase, wherein said catalyst is formed by passing a hydrocarbon-containing gas over a catalyst precursor at an elevated temperature for sufficient time to form the active carbon phase.

The chemical reaction is preferably a dehydrogenation reaction and the reactant is preferably a hydrocarbon, in particular an alkane. In a preferred process, the catalyst precursor comprises a metal compound. In an alternative embodiment of the invention the catalyst or catalyst precursor comprises a preformed carbon nanofibre material.

The elevated temperature is preferably at least 650° C., particularly between 650° C. and 750° C., especially greater than 670° C. and most preferably in the range from 670-730° C. We have found that the process is very satisfactory at a reaction temperature of about 700° C.

### BRIEF DESCRIPTION OF THE DRAWINGS

FIG. 1: a diagram showing the process used to perform dehydrogenation reactions described in the Examples.

FIG. 2: a graph showing conversion over time for a vanadia catalyst operated at different temperatures.

FIG. 3: a graph showing propylene yield over time for a vanadia catalyst operated at different temperatures.

FIG. 4: a graph showing conversion over time for various vanadia and iron catalysts operated at 700° C.

FIG. 5: a graph showing propylene yield over time for various vanadia and iron catalysts operated at 700° C.

FIG. 6: a graph showing conversion over time for a vanadia catalyst operated first at 700° C. and then at a temperature of 600, 625 or 650° C.

FIG. 7: a graph showing propylene yield over time for a vanadia catalyst operated first at 700° C. and then at a temperature of 600, 625 or 650° C.

FIG. 8: a graph showing propylene yield and conversion over time for a vanadia catalyst operated first at 700° C., then cooled, and then operated at a range of temperatures from 600° C.

### DETAILED DESCRIPTION OF THE INVENTION

According to a further aspect of the invention we provide a process for the dehydrogenation of a hydrocarbon comprising the step of contacting a feed stream containing said hydrocarbon with a catalyst comprising a metal compound or a carbon nanostructure at a temperature of at least 650° C., preferably between 650° C. and 750° C., especially in the range from 680-730° C., for example about 700° C. The feed stream containing the hydrocarbon is contacted with the catalyst for sufficient time at said temperature greater than 650° C. for carbon to form on the catalyst surface. Preferably sufficient carbon is formed on the catalyst so that at least 3%, more preferably at least 5% of the catalyst, by weight, comprises carbon formed by reaction of a hydrocarbon containing feed stream with the catalyst at said elevated temperature greater than 650° C. The process is preferably operated by contacting said feed stream with said catalyst or precursor at said elevated temperature for at least 1 hour, more preferably at least 3 hours, especially at least 6 hours. This contact enables an active phase of carbon to form on the catalyst.

The metal compound preferably comprises a transition metal compound, more especially a compound of a metal selected from V, Cr, Mn, Fe, Co, Mo, Ni, Au, Pt, Pd, Ru and Rh. The metal compound may comprise the metal in elemental form or it may be a compound such as an oxide (including mixed oxides where the metal forms more than one oxide), carbonate, nitrate, sulphate, sulphide or hydroxide. More than one metal compound may be present in the catalyst. In particular the catalyst may comprise a metal in more than one oxidation state, for example as a mixture of elemental metal and a metal oxide or more than one metal oxide. In a preferred form the metal compound comprises at least one oxide of the metal. A promoter metal may also be present in the catalyst. The metal compound may be supported or unsupported but is preferably supported on a porous support material. Suitable supports include silica, alumina, silica-alumina, titania, zirconia, ceria, magnesia and carbon. A preferred support is a transition alumina. A supported metal compound catalyst may be formed using any of the known methods such as precipitation, co-precipitation, deposition precipitation or impregnation of the support with a compound of the metal. This may be followed by calcination in an oxygen-containing gas at elevated temperature to form a metal oxide. The amount of metal in the catalyst varies according to the metal used. For example, we have found that when the metal is vanadium, the catalyst is most effective when it contains between 0.5% V and 5% V. Preferably the metal content is between 0.1% and 50%, more preferably between 0.1% and 10%, for example 0.5-10% and especially 0.5-5%.

WO 03/086625 describes a hydrocarbon dehydrogenation process using a catalyst composite comprising a Group VIII metal component, a Group IA or IIA metal component and a component selected from the group consisting of tin, germanium, lead, indium, gallium, thallium or mixtures thereof on a theta alumina support having a surface area of 50-120 m²/g, an apparent bulk density of at least 0.5 g/cm³ and a mole ratio of Group VIII noble metal component to the component selected from the group consisting of tin, germanium, lead, indium, gallium, thallium or mixtures thereof in the range from 1.5 to 1.7. The related US 2005/0033101 describes a similar process using a catalyst having the same metal components, surface area and bulk density as WO03/086625 but in which the mole ratio of the Group IA or IIA metal component to the component selected from the group consisting of tin, germanium, lead, indium, gallium, thallium or mixtures thereof is greater than about 16. In these documents the dehydrogenation process is described as endothermic and the feed stream is heated. Provision is made to reheat the feed stream by carrying out a selective oxidation reaction by introducing some oxygen in order to oxidize the hydrogen produced by dehydrogenating the hydrocarbon. By contrast, the process of the invention is a non-oxidative hydrogenation and is carried out in the absence of oxygen. It is preferred that neither the catalyst nor the catalyst precursor used in the method of the present invention comprise a catalyst composite comprising a Group VIII metal component, a Group IA or IIA metal component and a component selected from the group consisting of tin, germanium, lead, indium, gallium, thallium or mixtures thereof on a theta alumina support, particularly a catalyst composite as described in WO 03/086625 or US 2005/0033101. Preferably the catalyst or catalyst precursor does not contain both tin and platinum. Preferably the catalyst is not chlorinated prior to use.

It has been found by the inventors of the present invention that, at temperatures above about 650° C., certain carbon deposits form on the surface of the catalyst which, it is believed, may be catalytically active in the dehydrogenation of alkanes. The carbon may be graphitic, in the form of graphene layers and/or in the form of nanostructures such as nanofibres or nanotubes. The role of the carbon formed on the catalyst at temperatures greater than 650° C. is not known with certainty. For example, it is possible that the presence of the carbon modifies the catalyst surface in a way which is beneficial. For this reason, the invention is not limited to forms in which the carbon formed actively catalyses the dehydrogenation reaction, although it appears likely that the carbon has some catalytic function.

The process includes the step of contacting the hydrocarbon feed with the catalyst at a temperature of at least, and preferably greater than, 650° C., more preferably at least 675° C. We have found that when the temperature is operated at a temperature greater than 650° C. the conversion and selectivity reach a steady state after about 1-5 hours in which the conversion and selectivity change very little, or increase very slightly during a further period of at least 10 hours. We have operated a process for the dehydrogenation of propane according to the invention using a catalyst containing vanadia (3.5% V) successfully for more than 100 hours. The process may be operated continuously or semi-continuously. The upper limit of temperature depends on the process economics and the nature of the metal oxide and support (if present), wherein phase changes or sintering may occur if the temperature is raised above a certain point, this temperature being dependent on the identity and the form of the metal or support. Normally the process is operated below 850° C. and preferably below 750° C. We have found in the dehydrogenation of propane that, although the conversion is high at 750° C., the selectivity to propylene, and thus the yield of propylene is less at 750 than at 700° C. Preferably the process is operated at a temperature in the range 650-750° C., especially 680-720° C. The process may be operated below 650° C. following a period of operation at or above 650° C. for sufficient time for the active phase of the catalyst to form. When the process has not been operated at a temperature of at least 650° C., the catalyst deactivates with increasing time online. When the process is operated at a temperature of at least 650° C. and preferably greater than 650° C. as indicated above, we have found that, following an initial period of 1- about 6 hours (depending on the catalyst used) during which the conversion of the hydrocarbon feed falls, the catalyst then maintains its activity and in some cases, increases in activity over periods of several hours so that the requirement for catalyst regeneration is greatly reduced compared with prior art processes. The attainment of “steady state” operation during which both the conversion and yield of dehydrogenated hydrocarbon product remain stable or increase slowly is a feature of the process of the present invention. In the steady state operation of the process the conversion of hydrocarbon feed preferably does not decrease by more than 2% over a period of ten hours.

In a preferred process, the hydrocarbon comprises an alkane which is dehydrogenated to form an unsaturated compound, preferably an alkene. The alkane may be any alkane which is susceptible to dehydrogenation. Linear or branched alkanes may be dehydrogenated. Preferred alkanes have from 2 to 24 carbon atoms, especially 3-10 carbon atoms. The dehydrogenation of propane and n-butane are especially preferred reactions because of the commercial importance of their dehydrogenated products, i.e. propene, butenes and butadiene. The hydrocarbon may comprise other compounds which are susceptible to dehydrogenation, in particular compounds containing alkyl substituents such as ethylbenzene, for example.

The feed stream may contain an inert diluent such as nitrogen or another inert gas. When the process includes a recycle to the reactor, the feed stream may also contain some product compounds such as the alkene(s) formed, hydrogen and any co-products. In one form, the feed stream consists essentially of the reactant hydrocarbon, e.g. an alkane and optionally one or more of an inert gas, and one or more product compounds. Preferably the feed stream does not include more than a trace amount of oxygen. More preferably the process is operated substantially in the absence of oxygen. The process of the invention is not an oxidative dehydrogenation process.

The reactor and/or catalyst bed and/or the feed stream is heated to a temperature sufficient to provide the required reaction temperature. The heating is accomplished by providing heating means of a conventional type known to chemical process engineers.

A portion of the product formed in the process may be recycled to the reactor, with an appropriate heating step if required. The product stream is separated to remove hydrogen, before or after any recycle stream is taken. The products are then further separated into product alkenes and unreacted alkane feed and any by products are removed if required. The process is, however, more selective than some prior art dehydrogenation processes and so the separation train may be greatly reduced compared with that found on a typical prior art dehydrogenation plant, thereby saving on both capital and operating cost. This saving is additional to the reduction in cost realised from the higher conversion and selectivity which is possible using the process of the invention compared with known commercial processes, for example using promoted platinum catalyst at reaction temperatures less than 625° C. For example, known commercial processes typically operate at a conversion of less than 30%. The process of the present invention may be operated at a conversion of 50-60% so that the amount of the feed recycle may be greatly reduced, thus reducing the overall volumetric flow-rate and the associated equipment size.

According to a further aspect of the invention we provide a method of forming a catalyst comprising a form of carbon which is active for the dehydrogenation of alkanes, by contacting a catalyst precursor comprising a metal compound with a hydrocarbon at a temperature of at least, and preferably greater than, 650° C. We have found that the active carbon forms effectively when the catalyst precursor is contacted with the hydrocarbon at a temperature in the range 650-750° C. for at least 1 and preferably at least 3 hours. We therefore also provide a catalyst comprising a metal compound and a catalytically active form of carbon formed by the aforementioned process. The hydrocarbon is conveniently an alkane. In a preferred form of the process the hydrocarbon used to form the active catalyst comprises the alkane contained in a feed stream for a dehydrogenation reaction. The metal compounds and suitable support materials for the metal compounds, have been described above. The catalyst including the active carbon phase may be formed ex-situ or in-situ in the reactor in which it is to be used as a catalyst. It is a particular benefit that the catalyst may be formed in the reactor used for dehydrogenation by contact of a metal oxide precursor with a hydrocarbon at a temperature of at least 650° C. and then used to catalyse the dehydrogenation of said alkane.

A significant difference between the process of the invention and dehydrogenation processes known in the art is that the coke deposits formed in the dehydrogenation reaction are not removed through oxidation or other catalyst regeneration steps. In the process of the invention the coke formed in the reaction remains on the catalyst within the reactor. The coke formed at temperatures greater than 650° C. is believed to be catalytically active. Therefore the dehydrogenation process of the invention is operated in the absence of a catalyst regeneration step. Prior art catalyst regeneration usually involves oxidation of the coke deposited on the catalyst and this is typically carried out frequently, possibly more than once per hour of reaction time. It is a feature of the present invention that the process is preferably operated for more than 12 hours, especially more than 24 hours without catalyst regeneration.

According to a still further aspect of the invention, we provide a process for the non-oxidative dehydrogenation of a hydrocarbon comprising the step of contacting a feed stream containing at least one hydrocarbon with a catalyst comprising a form of carbon which is active for the dehydrogenation of alkanes. By non-oxidative dehydrogenation, we mean the dehydrogenation of alkanes in the absence of oxygen. Without wishing to be bound by theory, the active form of carbon is believed to be a structurally ordered deposit of carbon, possibly in the form of a nanostructure. By carbon nanostructure, we include nanofibres, nanotubes and other ordered nanoscale forms of carbon. The carbon nanostructure may be unsupported or supported. When supported, any conventional catalyst support may be used, including but not limited to carbon, silica, alumina, silica-alumina, titania, zirconia, ceria and magnesia in the form of granules, particles, fibres etc. A metal compound as described above may be present on the support. The catalyst may be formed by contacting a catalyst precursor comprising a metal compound with a hydrocarbon at a temperature of at least, and preferably greater than, 650° C. The hydrocarbon has been described above. In a preferred form of the invention the hydrocarbon comprises at least one alkane and the process is for dehydrogenation of the alkane to form an unsaturated compound, especially an alkene.

### EXAMPLES

The process will be demonstrated in the following examples and with reference to the accompanying drawings.

### Example 1

### Catalyst A

An aqueous solution of NH₄VO₃ (>99%, Aldrich) was prepared containing oxalic acid to ensure the dissolution of NH₄VO₃ [NH₄VO₃/oxalic acid=0.5 (molar ratio)]. The solution was used to impregnate an extruded θ-Al₂O₃ catalyst support having a BET surface area of 101 m² g⁻¹, and pore volume of 0.60 ml g⁻¹, using incipient wetness methodology. The solution used was calculated to provide a finished catalyst containing 1 wt % of vanadium. After impregnation the catalyst precursor was mixed thoroughly for 2 h at 77° C. to ensure a homogeneous distribution of vanadia on the support. The catalyst (designated Catalyst A) was then dried in air at 120° C. overnight and calcined in air for 6 h at 550° C. Analysis of Catalyst A by X-ray fluorescence (XRF) found 0.80% V by weight.

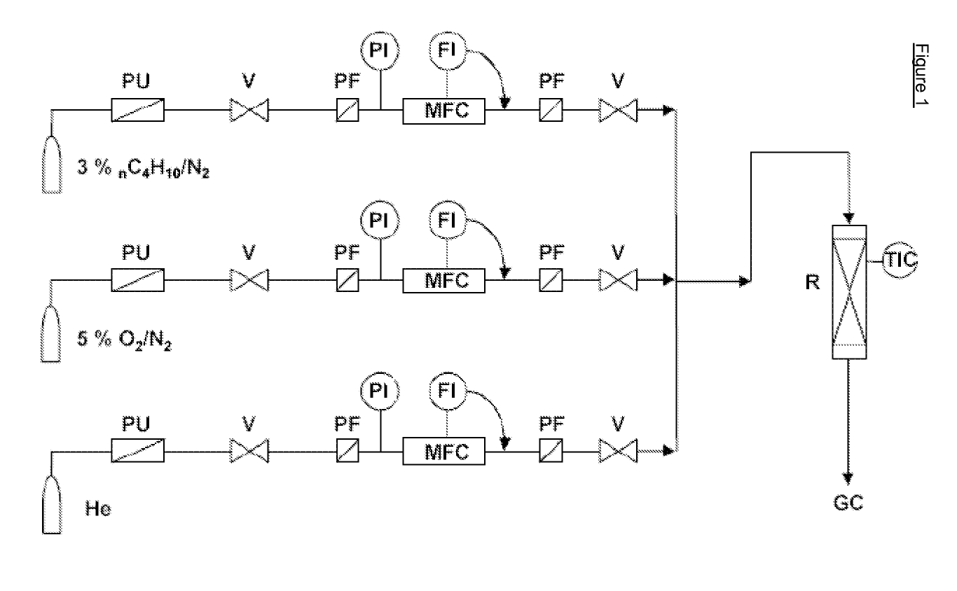

Catalytic activity data were acquired using a fixed-bed, continuous flow reactor quartz reactor (350 mm×12 mm o.d.) connected to an on-line gas chromatography (GC) instrument (Agilent 6890 Series-FID, using Agilent HP-5 column), as illustrated in FIG. 1. Prior to use the catalyst extrudates were ground and sieved to a particle size of 75-90 μm. The catalyst (2.6 cm³) was heated (5° C. min-1) to 700° C. in 5% O₂/N₂ (0.5 barg, 40 ml min-1) and held at this temperature for 2 h. A flow of He (0.5 barg, 42 ml min-1) was then established and the temperature adjusted to reaction temperature set-point of 700° C. (measured at 690° C.) and held at this temperature to stabilise for at least 30 min. 3% n-butane in N₂ was then introduced (0.5 barg, 60 ml min⁻¹) for a period of 3 h. GC measurements were taken at regular intervals and the gas phase composition of the effluent is shown in Table 1. After 3 h the catalyst was cooled to room temperature in flowing He and removed for ex situ analysis.

TABLE 1

| | Gas Phase Composition (%) | | | | | | |
|---|---|---|---|---|---|---|---|
| Time | Cracking | | | other | | | Higher |
| (min) | products | 1-butene | 1,3-butadiene | diene | n-butane | 2-butenes | hydrocarbons |
| 5 | 0.41 | 99.06 | 0.00 | 0.00 | 0.53 | 0.00 | 0.00 |
| 30 | 0.08 | 91.23 | 0.00 | 0.00 | 0.03 | 0.00 | 8.66 |
| 55 | 0.15 | 90.90 | 0.00 | 0.00 | 0.02 | 0.00 | 8.93 |
| 80 | 0.21 | 91.14 | 0.00 | 0.00 | 0.02 | 0.00 | 8.63 |
| 110 | 0.26 | 91.71 | 0.00 | 0.00 | 0.02 | 0.00 | 8.01 |
| 130 | 0.32 | 91.18 | 0.00 | 0.00 | 0.03 | 0.00 | 8.47 |
| 150 | 0.43 | 91.36 | 0.00 | 0.00 | 0.03 | 0.00 | 8.18 |
| 180 | 0.47 | 91.62 | 0.00 | 0.00 | 0.06 | 0.00 | 7.86 |

### Example 2

### Catalyst B

Vanadia on alumina catalysts calculated to contain 3.5% V by weight were made using the methods described in Example 1 by varying the concentration of the NH₄VO₃ solution. The catalyst (Catalyst B) was found to contain 3.68% V on analysis by XRF.

Catalyst B was tested in the dehydrogenation of butane, as described in Example 1. The effluent gas phase compositions are shown in Table 2.

TABLE 2

| | Gas Phase Composition (%) | | | | | | |
|---|---|---|---|---|---|---|---|
| Time | Cracking | | | other | | | Higher |
| (min) | products | 1-butene | 1,3-butadiene | diene | n-butane | 2-butenes | hydrocarbons |
| 5 | 0.00 | 86.96 | 0.00 | 0.00 | 0.00 | 0.00 | 13.04 |
| 30 | 1.13 | 47.32 | 11.10 | 0.00 | 35.94 | 0.72 | 3.79 |
| 110 | 0.04 | 26.46 | 16.98 | 0.20 | 55.46 | 0.62 | 0.24 |
| 180 | 0.02 | 26.33 | 17.50 | 0.19 | 55.14 | 0.66 | 0.16 |

### Example 3

### Catalyst C

Vanadia on alumina catalysts containing a nominal 8% V by weight were made and tested using the methods described in Example 1 by varying the concentration of the NH₄VO₃ solution. Analysis of the catalyst (Catalyst C) by XRF found 7.9% V by weight. The effluent gas phase compositions from the dehydrogenation reaction are shown in Table 3.

Catalysts A, B & C were removed from the reactor and examined by microanalysis to determine the amount of carbon formed during the reaction. The results are shown in Table 4 and suggest that the very high conversion and selectivity to 1-butene formation at 690° C. using Catalyst A may be due to the significantly greater weight of carbon which is formed on this catalyst under reaction conditions.

TABLE 3

| | Gas Phase Composition (%) | | | | | | |
|---|---|---|---|---|---|---|---|
| Time | Cracking | | | other | | | Higher |
| (min) | products | 1-butene | 1,3-butadiene | diene | n-butane | 2-butenes | hydrocarbons |
| 5 | 0.00 | 95.65 | 3.60 | 0.30 | 0.00 | 0.05 | 0.42 |
| 30 | 0.68 | 28.33 | 13.45 | 0.20 | 55.96 | 0.63 | 0.75 |
| 110 | 0.00 | 23.70 | 16.51 | 0.22 | 58.91 | 0.50 | 0.17 |
| 180 | 0.00 | 23.78 | 16.72 | 0.21 | 58.66 | 0.46 | 0.15 |

TABLE 4

| | Amount of C |
|---|---|
| Catalyst | (wt %) |
| A | 6.67 |
| B | 2.25 |
| C | 3.58 |
| Al₂O₃ support | 0.96 |

### Example 4

A fresh sample of Catalyst B was tested in the dehydrogenation of butane using the reaction described in Example 1 at a reaction set-point temperature of 675° C. (actual temperature approx. 665° C.). The results are shown in Table 5.

TABLE 5

| | Gas Phase Composition (%) | | | | | | |
|---|---|---|---|---|---|---|---|
| Time | Cracking | | | other | | | Higher |
| (min) | products | 1-butene | 1,3-butadiene | diene? | n-butane | 2-butenes | hydrocarbons |
| 5 | 0.00 | 81.48 | 12.89 | 4.56 | 0.48 | 0.60 | 0.00 |
| 30 | 0.65 | 18.56 | 8.22 | 0.19 | 69.42 | 1.43 | 1.54 |
| 110 | 0.00 | 11.50 | 8.77 | 0.23 | 78.78 | 0.59 | 0.13 |
| 180 | 0.00 | 10.93 | 8.85 | 0.23 | 79.23 | 0.69 | 0.07 |

### Comparative Examples 5 & 6

Fresh samples of Catalyst B were tested in the dehydrogenation of butane using the reaction described in Example 1 at measured temperatures of 625 and 550° C. The results are shown in Tables 6 & 7 respectively. Examples 2 and 4-6 show that at temperatures greater than 650° C. the conversion of n-butane and selectivity to 1-butene as a product are significantly greater than at lower temperatures. Tables 6 and 7 show that the yield of C4 products (butenes and butadienes) decreases with increasing time on stream at temperatures of 625° C. and below and remains relatively stable or increases at the higher temperatures used in Examples 2 and 4.

TABLE 6

| | Gas Phase Composition (%) | | | | | | |
|---|---|---|---|---|---|---|---|
| Time | Cracking | | | | | | Higher |
| (min) | products | 1-butene | 1,3-butadiene | other | n-butane | 2-butenes | hydrocarbons |
| 5 | 0.00 | 28.73 | 13.42 | 0.31 | 46.85 | 6.65 | 4.03 |
| 30 | 0.58 | 8.20 | 4.21 | 0.18 | 84.58 | 1.53 | 0.74 |
| 110 | 0.00 | 3.88 | 2.59 | 0.23 | 92.37 | 0.82 | 0.11 |
| 180 | 0.00 | 3.20 | 2.42 | 0.24 | 93.49 | 0.61 | 0.04 |

TABLE 7

| | Gas Phase Composition (%) | | | | | | |
|---|---|---|---|---|---|---|---|
| Time | Cracking | | | | | | Higher |
| (min) | products | 1-butene | 1,3-butadiene | other | n-butane | 2-butenes | hydrocarbons |
| 5 | 3.20 | 2.93 | 0.22 | 89.28 | 3.61 | 0.04 | 0.72 |
| 30 | 0.39 | 2.41 | 2.01 | 0.19 | 90.66 | 3.75 | 0.58 |
| 110 | 0.00 | 1.09 | 0.85 | 0.21 | 95.88 | 1.72 | 0.25 |
| 150 | 0.00 | 0.93 | 0.74 | 0.22 | 96.81 | 1.15 | 0.15 |
| 180 | 0.00 | 0.87 | 0.70 | 0.22 | 96.93 | 1.14 | 0.14 |

### Example 7

Example 2 was repeated with the exception that the catalyst sample was calcined in the 5% O₂/N₂ gas mixture at 550° C. instead of 700° C. The reaction temperature set-point was 700° C. The results are shown in Table 8. The lower calcination temperature appears to result in a small decrease in the conversion which is stable after about 1 hour at about 44%, compared with a conversion of about 50% when the catalyst was calcined at 700° C.

TABLE 8

| | | Gas Phase Composition (%) | | | | | | |
|---|---|---|---|---|---|---|---|---|
| Time | | Cracking | | 1,3- | | | | Higher |
| (min) | Conversion | products | 1-butene | butadiene | other | n-butane | 2-butenes | hydrocarbons |
| 5 | 100.00 | 0.00 | 100.00 | 0.00 | 0.00 | 0.00 | 0.00 | 0.00 |
| 30 | 55.95 | 0.52 | 28.71 | 12.03 | 0.18 | 56.32 | 0.66 | 1.56 |
| 55 | 48.20 | 0.10 | 22.06 | 13.63 | 0.21 | 63.03 | 0.65 | 0.34 |
| 130 | 44.08 | 0.00 | 20.78 | 15.01 | 0.21 | 63.14 | 0.75 | 0.12 |
| 180 | 44.23 | 0.00 | 20.96 | 15.43 | 0.21 | 62.64 | 0.63 | 0.13 |

### Example 8

Example 1, i.e. using Catalyst A, was repeated with the exception that the feed stream for the dehydrogenation reaction was 100% butane, rather than the 3% n-butane in N₂ used in Example 1. The results are shown below in Table 9. After approximately 30 minutes, the conversion is maintained at about 95%.

TABLE 9

| | Gas Phase Composition (%) | | | | | | |
|---|---|---|---|---|---|---|---|
| Time | Cracking | | | other | | | Higher |
| (min) | products | 1-butene | 1,3-butadiene | diene? | n-butane | 2-butenes | hydrocarbons |
| 5 | 0.07 | 94.05 | 3.32 | 0.00 | 0.32 | 0.05 | 2.19 |
| 30 | 1.27 | 67.96 | 17.03 | 0.00 | 8.98 | 0.81 | 3.95 |
| 110 | 0.99 | 61.81 | 22.64 | 0.00 | 10.39 | 0.84 | 3.32 |
| 180 | 0.97 | 59.66 | 23.18 | 0.00 | 11.89 | 0.96 | 3.34 |

### Example 9

Example 2, i.e. using Catalyst B, was repeated with the exception that the feed stream for the dehydrogenation reaction was 100% butane, rather than the 3% n-butane in N₂ used in Example 2. The results are shown below in Table 10. After approximately 30 minutes, the conversion is maintained at about 95%.

TABLE 10

| | Gas Phase Composition (%) | | | | | | |
|---|---|---|---|---|---|---|---|
| Time | Cracking | | | other | | | Higher |
| (min) | products | 1-butene | 1,3-butadiene | diene? | n-butane | 2-butenes | hydrocarbons |
| 5 | 0.00 | 94.34 | 1.00 | 0.00 | 0.22 | 0.03 | 4.40 |
| 30 | 0.00 | 60.51 | 22.15 | 0.00 | 12.85 | 0.37 | 4.12 |
| 110 | 0.00 | 60.16 | 23.17 | 0.00 | 12.22 | 0.41 | 4.05 |
| 180 | 0.00 | 58.75 | 23.42 | 0.00 | 13.32 | 0.44 | 4.07 |

### Example 10

The dehydrogenation reaction described in Example 1, including calcination at 700° C., was operated using a commercially available catalyst comprising 0.5% platinum supported on a shaped alumina support. The results, shown in Table 11, indicate that the reaction does not maintain a steady conversion during the experiment although the conversion is relatively high. This may be due to the activity of reduced platinum as a catalyst for the hydrogenation of olefins and diolefins.

TABLE 11

| | Gas Phase Composition (%) | | | | | | |
|---|---|---|---|---|---|---|---|
| Time | Cracking | | 1,3- | | | | Higher |
| (min) | products | 1-butene | butadiene | other | n-butane | 2-butenes | hydrocarbons |
| 5 | 0.00 | 94.53 | 0.00 | 0.00 | 0.00 | 0.00 | 5.47 |
| 30 | 0.00 | 81.05 | 3.07 | 0.00 | 1.10 | 0.04 | 14.74 |
| 110 | 0.00 | 78.86 | 8.81 | 0.00 | 5.43 | 0.19 | 6.72 |
| 180 | 0.00 | 70.86 | 11.89 | 0.00 | 12.47 | 0.36 | 4.42 |

### Example 11

The dehydrogenation reaction, including calcination at 700° C., described in Example 1 was operated using a commercially available catalyst comprising 0.3% palladium supported on a shaped alumina support. The results, shown in Table 12, indicate that conversion is steady at 100% with a very high selectivity to 1-butene.

TABLE 12

| | Gas Phase Composition (%) | | | | | | |
|---|---|---|---|---|---|---|---|
| Time | Cracking | | 1,3- | other | | | Higher |
| (Min) | products | 1-butene | butadiene | diene? | n-butane | 2-butenes | hydrocarbons |
| 5 | 0.00 | 100.00 | 0.00 | 0.00 | 0.00 | 0.00 | 0.00 |
| 30 | 0.00 | 95.37 | 0.00 | 0.00 | 0.00 | 0.00 | 4.63 |
| 110 | 0.00 | 95.33 | 0.00 | 0.00 | 0.00 | 0.00 | 4.67 |
| 180 | 0.00 | 95.64 | 0.00 | 0.00 | 0.00 | 0.00 | 4.36 |

### Example 12

The dehydrogenation reaction, including calcination at 700° C., described in Example 1 was operated using a commercially available catalyst comprising 35% iron supported on alumina. The results, shown in Table 13, indicate that conversion is steady at >99% with a very high selectivity to 1-butene.

TABLE 13

| | Gas Phase Composition (%) | | | | | | |
|---|---|---|---|---|---|---|---|
| Time | Cracking | | 1,3- | other | | | Higher |
| (Min) | products | 1-butene | butadiene | diene? | n-butane | 2-butenes | hydrocarbons |
| 5 | 15.51 | 84.22 | 0.00 | 0.00 | 0.26 | 0.00 | 0.00 |
| 30 | 0.23 | 99.08 | 0.00 | 0.00 | 0.47 | 0.00 | 0.00 |
| 110 | 0.78 | 98.94 | 0.00 | 0.00 | 0.27 | 0.00 | 0.00 |
| 180 | 2.25 | 96.22 | 0.00 | 0.00 | 1.53 | 0.00 | 0.00 |

### Example 13

The dehydrogenation reaction, including calcination at 700° C., described in Example 1 was operated using a commercially manufactured, unsupported carbon nanofibre, PYROGRAF™ III, type PR24XT-LHT, supplied by Applied Sciences Inc. The results are shown in Table 14, below.

TABLE 14

| | Gas Phase Composition (%) | | | | | | |
|---|---|---|---|---|---|---|---|
| Time | Cracking | | 1,3- | other | | | Higher |
| (Min) | products | 1-butene | butadiene | diene? | n-butane | 2-butenes | hydrocarbons |
| 5 | 0.00 | 74.62 | 12.63 | 0.00 | 12.50 | 0.25 | 0.00 |
| 30 | 0.00 | 51.72 | 18.54 | 0.00 | 27.00 | 0.20 | 2.54 |
| 55 | 0.16 | 51.36 | 18.47 | 0.00 | 26.61 | 0.17 | 3.23 |
| 80 | 0.42 | 51.10 | 18.42 | 0.00 | 26.57 | 0.13 | 3.34 |
| 110 | 0.43 | 51.21 | 18.38 | 0.00 | 26.46 | 0.15 | 3.37 |
| 130 | 0.43 | 51.25 | 18.36 | 0.00 | 26.41 | 0.15 | 3.40 |
| 150 | 0.43 | 51.07 | 18.42 | 0.00 | 26.49 | 0.16 | 3.43 |
| 180 | 0.42 | 50.99 | 18.40 | 0.00 | 26.61 | 0.12 | 3.46 |

### Example 14

Example 1 was repeated using a feed gas for the dehydrogenation consisting of 100% propane instead of the 3% n-butane in N₂ mixture. The results are shown in Table 15, below and indicate that the process is stable and highly effective for the dehydrogenation of propane.

TABLE 15

Gas Phase Composition (%)

| Time | Cracking | | | Other |
|---|---|---|---|---|
| (Min) | products | propene | propane | hydrocarbons |
| 5 | 0.00 | 100.00 | 0.00 | 0.00 |
| 30 | 0.00 | 100.00 | 0.00 | 0.00 |
| 55 | 0.00 | 87.99 | 8.65 | 3.17 |
| 80 | 0.00 | 83.98 | 11.69 | 4.08 |
| 110 | 0.00 | 77.55 | 16.98 | 5.09 |
| 130 | 0.00 | 74.62 | 19.35 | 5.58 |
| 150 | 0.00 | 72.89 | 21.00 | 5.62 |
| 180 | 0.00 | 71.60 | 22.15 | 5.69 |

### Example 15

A catalyst containing 3.2 wt % of V (by XRF) was made by impregnating particles of an extruded theta Al₂O₃ catalyst support in the form of trilobes with an aqueous solution of NH₄VO₃ as described in Example 1, but by tumbling the catalyst support for 2 h at room temperature instead of at 77° C. The catalyst was calcined as described in Example 1.

Catalytic activity data were acquired using a fixed-bed, continuous flow high temperature stainless steel reactor (1000 mm×18 mm i.d.) connected to an on-line gas chromatography (GC) instrument. The catalyst (9 cm³) was heated (5° C. min⁻¹) to 700° C. in 5% O₂/N₂ (0.5 barg, 140 ml min⁻¹) and held at this temperature for 2 hours. A flow of N₂ (1 barg, 193 ml min⁻¹) was then established and the temperature adjusted to the required reaction temperature and held at this temperature to stabilise for at least 30 min. 3.6% propane (7 ml min⁻¹) in N₂ was then introduced (total flow 1 barg, 200 ml min⁻¹). GC measurements were taken at regular intervals to determine the gas phase composition (propane, propene, methane, ethane and ethane). At the end of the run the propane flow was stopped and the catalyst was allowed to cool to room temperature under a flow of N₂ (1 barg, 193 ml min⁻¹).

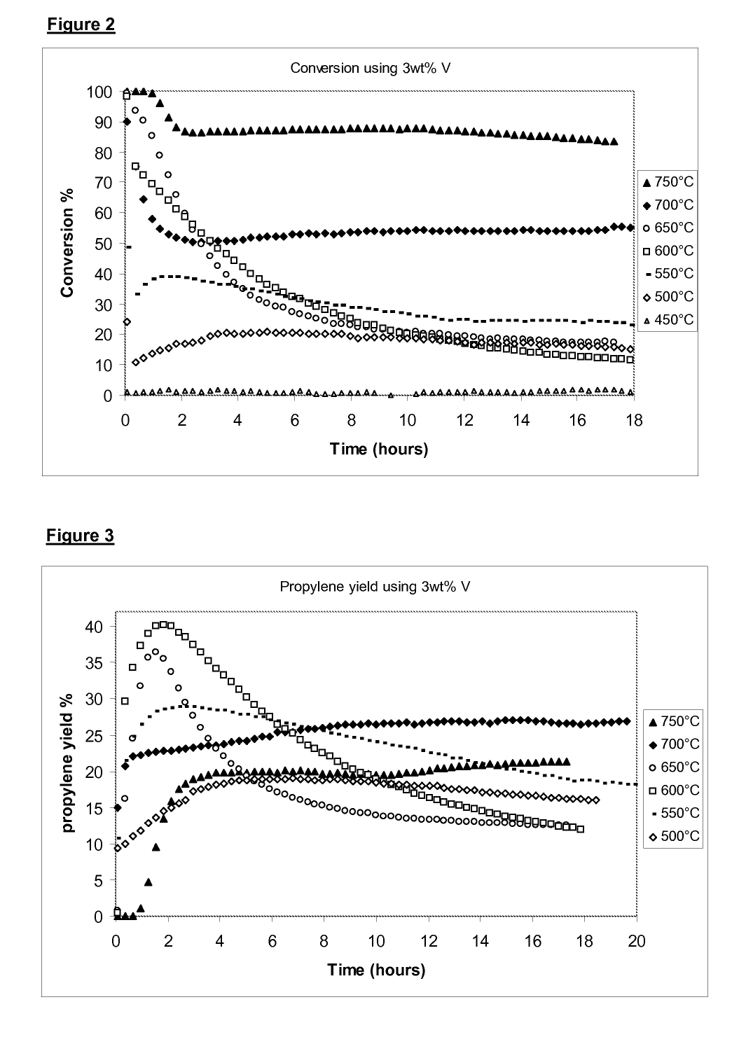

In separate runs, the process was operated at a steady-state at the following temperatures: −450, 500, 550, 600, 650, 700 and 750° C. The propane conversion and propylene yield were calculated using the following method and these are shown in FIGS. 2 and 3.

Propane conversion(%)=(1−[propane out]/[propane in])*100

Propylene yield(%)=100*[propylene out]/[propane in]

Although the steady-state conversion at 750° C. is higher than that at 700° C., the amount of cracked products seen in this reactor at 750° C. was significantly higher than at 700° C. The steady-state propylene yield is maximized at 700° C. By “steady-state” we mean the state of the reaction after continuous operation for at least two hours after which the reaction, as characterised by conversion, for example, does not appear to change significantly. This is believed to be the period following the formation of the active carbon phase of the catalyst.

### Example 16

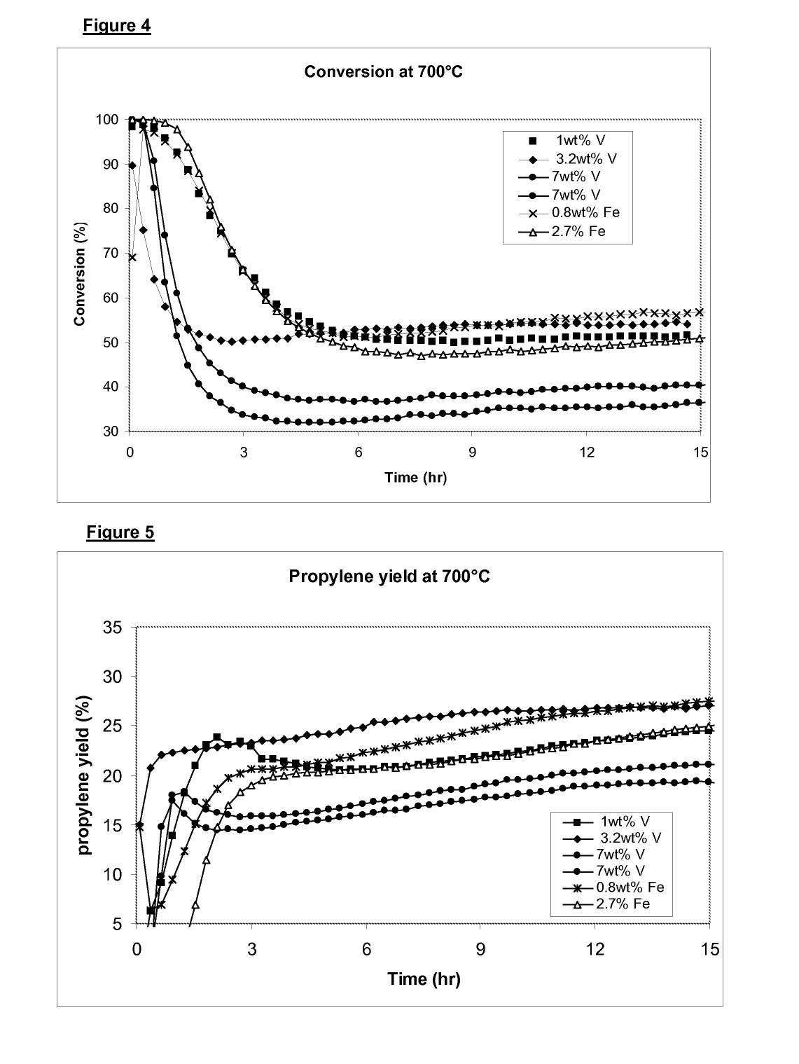

Catalysts consisting of different amounts of metal compounds on alumina trilobes were prepared and used in the dehydrogenation of propane as described in Example 15 using a reaction temperature of 700° C. The catalysts used contained, as metals, vanadium (1.0%, 3.2%, 7.0%) and iron (0.8% and 2.7%). The propane conversion and propylene yield are shown in FIGS. 4 and 5. The results indicate that, following an initial period of time during which the conversion decreases and the propylene yield increases, each of the reactions using the catalysts tested attains a “steady state” during which both the conversion and yield remain stable or increase slowly. This steady state has been found to persist for more than 4 days when the reaction has been allowed to proceed. The 3.2% V catalyst achieved steady state operation more quickly than the other catalysts.

### Example 17

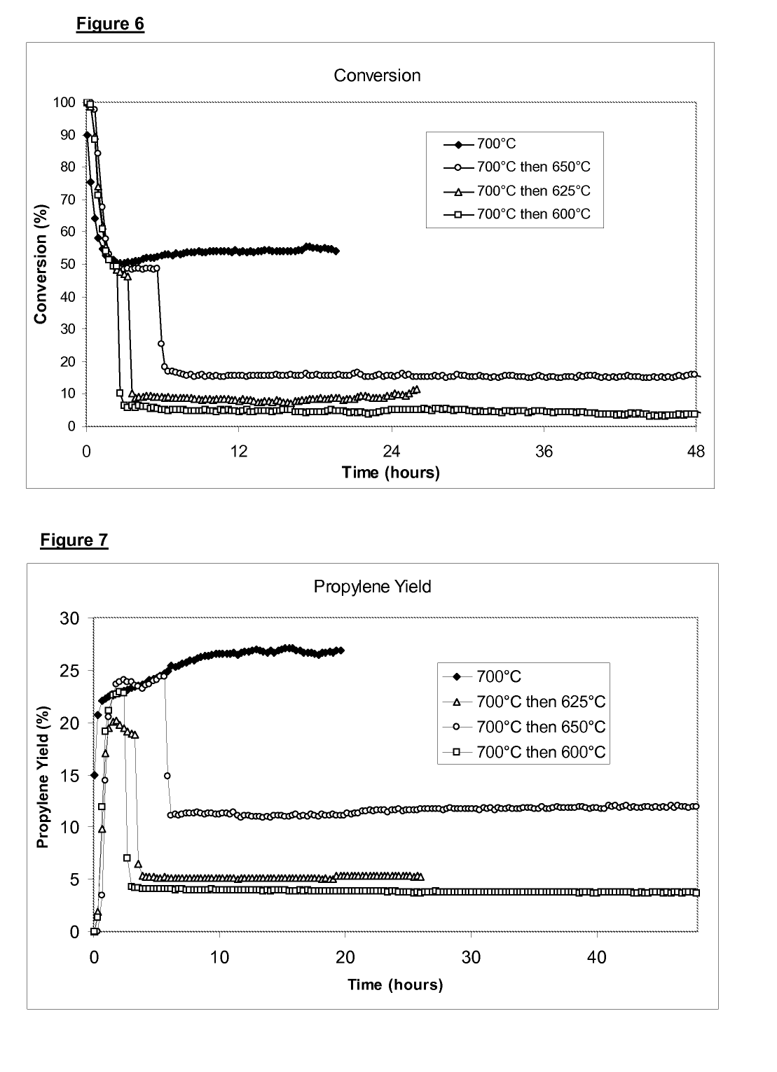

Further samples of the catalyst made in Example 15 were used in the dehydrogenation of propane as described in Example 16, with the exception that after operation at 700° C. for about 3-5 hours (a time indicated by the rapid decrease in conversion shown in FIGS. 6 and 7), the temperature of the reactor was reduced to 650, 625 or 600° C. The results are shown in FIGS. 6 and 7, together with the data from FIGS. 2 and 3 for the 700° C. run. The results show that, compared with operation at a continuous temperature of 650 and 600° C. (shown in FIGS. 2 and 3), steady state operation is achieved more rapidly by first operating the reaction at 700° C. The reaction at 650° C. was continued successfully for more than 100 hours. The propylene yield at 116 hours was 12%. The average propylene yield between 10 hours and 15 hours was 11.1% and the average propylene yield between 100 hours and 105 hours was 11.9%.

### Example 18

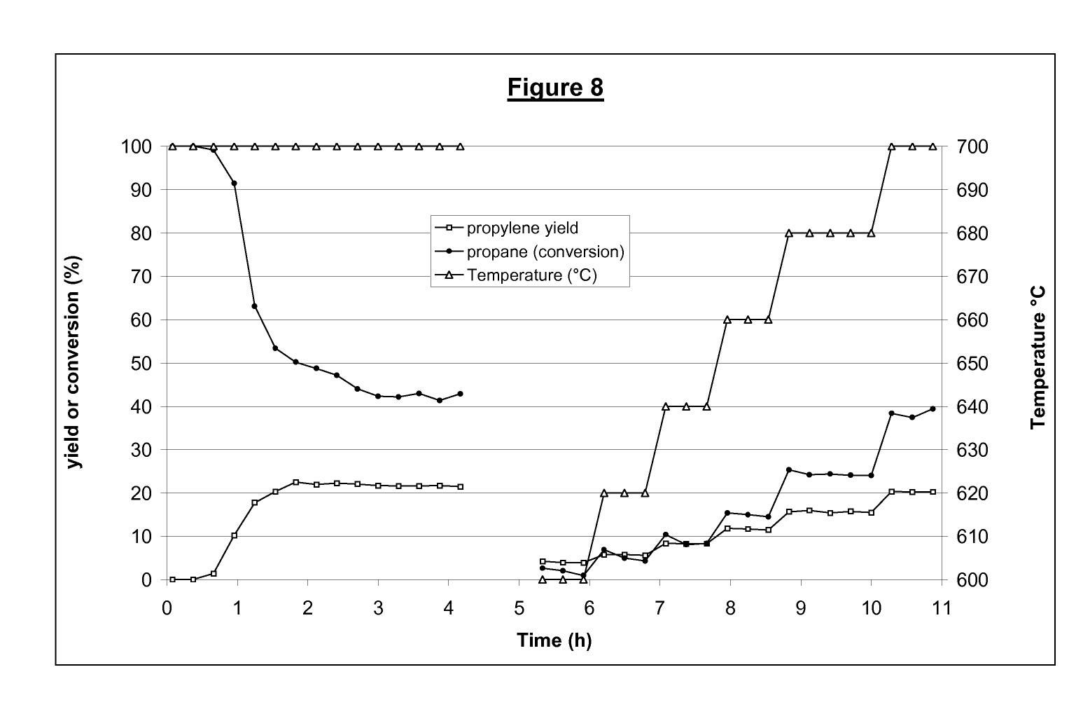

A sample of catalyst containing 3.5% of V on particles of alumina trilobe support was used in the dehydrogenation process described in Example 15 using a reaction temperature of 700° C. After about 4 hours, the propane supply was stopped and the catalyst allowed to cool down under nitrogen (193 ml/min). The catalyst was taken out of the reactor and the amount of carbon, as measured by pyrolysis and infra-red detection using a LECO™ carbon analyser, was found to be 9.6%. The catalyst was then put back into the reactor, a flow of nitrogen (193 ml/min) was started and the temperature raised to 600° C. After 15 minutes stabilisation at 600° C. the flow of propane was turned on (7.4 ml/min). The gas composition was analysed by GC, the temperature was then raised to 620, 640, 660, 680 and then 700° C. The conversion and propylene yield at each temperature is shown in FIG. 8.

### Example 19

A process was operated as described in Example 15 at 700° C. using a catalyst containing vanadia (3.5% V). A sample of the catalyst removed after 3 hours was found to contain about 10% by weight of carbon. A sample of the catalyst removed after 6 hours was found to contain about 11% by weight of carbon.

1. A process for dehydrogenation of a hydrocarbon comprising the step of passing a feed stream containing at least one hydrocarbon over a catalyst comprising a catalytically active carbon phase, wherein said catalyst is formed by passing a hydrocarbon-containing gas over a catalyst precursor at an elevated temperature for sufficient time to form the active carbon phase.

2. A process according to claim 1, wherein said catalyst precursor comprises a metal compound.

3. A process according to claim 2, wherein said metal compound is a compound of a metal selected from the group consisting of V, Cr, Mn, Fe, Co, Ni, Pt, Pd, Ru, Au, Mo and Rh.

4. A process according to claim 2, wherein the metal compound comprises the metal in elemental form or an oxide, carbonate, nitrate, sulphate, sulphide or hydroxide of the metal.

5. A process according to claim 2, wherein said metal compound is supported on a porous support material.

6. A process according to claim 1, wherein said catalyst precursor comprises a preformed carbon nanofibre material.

7. A process according to claim 1, wherein said hydrocarbon comprises an alkane having from 2 to 24 carbon atoms and which is dehydrogenated to form an alkene.

8. A process according to claim 1, wherein said dehydrogenation proceeds substantially in the absence of oxygen.

9. A process according to claim 1, wherein said elevated temperature is in the range from 650-750° C.

10. A process as claimed in claim 1, wherein said hydrocarbon-containing gas is passed over said catalyst precursor at said elevated temperature for at least one hour.

11. A method of forming a catalyst for the dehydrogenation of alkanes, comprising the step of contacting a catalyst precursor with a hydrocarbon at a temperature greater than 650° C.

12. A method according to claim 11, wherein said catalyst precursor comprises a compound of a metal or a preformed carbon nanofibre.

13. A method according to claim 12, wherein said metal is selected from the group consisting of V, Cr, Mn, Fe, Co, Ni, Pt, Pd, Ru and Rh.

14. A method according to claim 12, wherein said metal compound comprises a metal oxide.

15. A method according to claim 12, wherein said metal compound is supported on a porous support material.

16. A method according to claim 11, wherein said hydrocarbon comprises an alkane and the catalyst is formed in-situ in a reactor suitable for carrying out a non-oxidative dehydrogenation of said alkane and further comprising the step of using said catalyst for catalysing the dehydrogenation of said alkane in said reactor.

17. A method for the non-oxidative dehydrogenation of an alkane to form an alkene comprising the step of contacting a feed stream containing at least one alkane with a catalyst comprising carbon in the form of a nanostructure.
